(19) United States
(12) Patent Application Publication
(10) Pub. No.: US 2018/0214501 A1
(43) Pub. Date: Aug. 2, 2018

(54) Colloidal suspension for the healing of various cancers

(71) Applicant: Shajar Abid, Mcleansville, NC (US)

(72) Inventor: Shajar Abid, Mcleansville, NC (US)

(21) Appl. No.: 15/418,347

(22) Filed: Jan. 27, 2017

(51) Int. Cl. A61K 36/71; A61K 35/644; A61K 36/28

(52) U.S. Cl. CPC A61K 36/71; A61K 36/28; A61K 35/644

(57) ABSTRACT

The present invention pertains to a herbal formulation with potent antineoplastic properties, in humans and animals. The composition consists of the powdered radix of saussurea lappa and nigella sativa in raw honey colloidal suspension in well-defined ratios. The invention also includes a process for preparing the formulation, by creating a honey based colloidal suspension of the components, nigella sativa and saussurea lappa.

### BACKGROUND OF THE INVENTION

The desire for natural cures is increasing, people are looking for herbs which can heal them. Many herbs have been known to cultures in South Asia for thousands of years, specifically in the Kashmir Valley of the Himalayas in Pakistan and yet remain unknown to western science.

Despite the costly chemotherapies and radiation systems, cancer patients are not being cured instead left with toxic side effects. Estimated 20 million americans are diagnosed with cancer. In 2016, an estimated 1,685,210 new cases of cancer were diagnosed in the United States and 595,690 people died from the disease. This leaves much to be desired. Thus the need in terms of a new methodology of using natural compounds to assist healing in a non-toxic way.

The present invention provides a novel herbal composition comprising powdered radix of saussurea lappa as well solid or powdered nigella sativa in colloidal suspension of natural honey. The composition which is meant for dietary consumption, exhibits remarkable antibiotic and tumor healing properties. It is particularly effective in treating and curing cancers, which are regarded as ‘incurable’ in state of the art. The potency and powerful antimicrobial and antineoplastic action of the herbal formulation is due to the synergistic action of the components, mainly plant extracts, present in it.

The present invention provides an effective and low cost method for treatment of various cancers.

### FIELD OF INVENTION

The present invention relates to the field of herbal formulations having therapeutic properties. More specifically, it relates to a herbal formulation having potent tumor healing properties, which is quite effective in curing cancers of various natures in humans. The herbal formulation is also quite effective in treating tumors in animals.

### SUMMARY OF THE INVENTION

According to the present invention a novel herbal formulation is provided for dietary consumption, for the treatment and cure of all types of cancers in humans and animals. The herbal formulation consists of powdered root of saussurea lappa, in a mixture of nigella sativa along with natural honey, in well-defined ratios. The composition exhibits remarkable efficacy in treating and curing tumors, which are regarded as ‘incurable’ in state of the art. The herbal formulation was arrived at by carrying out meticulous studies by the inventor, to identify the most effective and active combination of plant extracts which can induce apoptosis naturally and without toxic side effects.

### OBJECTS OF THE INVENTION

It is a general objective of the invention to provide a novel herbal formulation for cancer cells apoptosis and its method of manufacture. Yet another objective is to provide a herbal formulation which provides an inexpensive alternative cancer healing therapy, which does not have undesirable side-effects. Another objective of the present invention is to disclose a herbal formulation which is easy to manufacture.

### DETAILED DESCRIPTION OF THE PREFERRED EMBODIMENT

The invention describes a herbal cancer healing formulation which is used to treat different types of tumor in humans and animals and helps in preventing the need for radiation and chemotherapy and mitigates their harmful effects.

The appropriate part of the herb used in the formulation is given below:

Part Used in

S. No. Scientific Name formulation

1. Nigella Sativa Seed

2. Saussurea lappa Radix

3. Grade A—Raw, Unfiltered Honey

It will be appreciated by those skilled in the art that the above approximate weight percents are dependent generally on the expected potencies of the individual components, whereby the relative weight percents will vary sometimes substantially from the above individual amounts. It will be within the skilled person's knowledge with this disclosure that the objects of the present invention require the inclusion of each of the components in relative mass ratios above.

The easy commercial availability of all the ingredients, the ease of manufacturing of the extracts and the finished product provides a economical and effective alternative cancer healing therapy, which does not have undesirable side-effects.

### Example 1 Preparation of Nigella Sativa

The seed of Nigella Sativa is weighed, then thoroughly washed in cold water to remove any dirt, soil, undesirable contaminants etc. Distilled or deionized water is then used to bring the seeds to a boil. The mixture is then boiled vigorously to sterilize and then drained. The filtrate is heated and then dried and filtered to remove the unwanted insoluble material. The dried herb is then grinded to obtain a dry pure powder.

The radix of Saussurea lappa is weighed, then thoroughly washed in cold water to remove any dirt, soil, undesirable contaminants etc. Distilled or deionized water is then used to bring the roots to a boil. The mixture is then boiled vigorously to sterilize. The mixture is then filtered to remove the unwanted insoluble material and dried. The dried herb is then grinded to obtain a dry pure powder.

### Preparation of the Formulation (Colloidal Suspension):

The two herbs as listed above in dry form are weighed in appropriate quantities and then thoroughly sterilized in boiling water. These are then dried and powdered. The dry weight of each herb is measured for the two herbs, Saussurea Lappa and Nigella Sativa. In equal ratios, mixed in a container are the dried herbs in powdered form, and added to natural (raw) honey.

I claim:

1) A colloidal suspension for healing tumors naturally comprising:
a) honey
b) radix of saussurea lappa
c) nigella sativa

2. The composition of claim 1, wherein weight ratios of any two components range from 1:5 to 5:1.

3. The composition of claim 2, wherein weight ratios of any two components range from 1:3 to 3:1.

4. The composition of claim 3, wherein weight ratios of any two components are 1:1.

5. The composition of claim 4, wherein weight ratios of any two components are 1:10 to 10:1
